(19) United States
(12) Patent Application Publication
(10) Pub. No.: US 2026/0196326 A1
(43) Pub. Date: Jul. 9, 2026

(54) DYNAMIC TAILORING OF A PRESCRIPTION TRANSACTION

(71) Applicants: Stanley Crane, San Marcos, CA (US); Thomas Borzilleri, San Marcos, CA (US)

(72) Inventors: Stanley Crane, San Marcos, CA (US); Thomas Borzilleri, San Marcos, CA (US)

(21) Appl. No.: 19/557,601

(22) Filed: Mar. 5, 2026

Related U.S. Application Data
(63) Continuation-in-part of application No. 17/398,915, filed on Aug. 10, 2021.

(51) Int. Cl. G16H 20/10; G06Q 10/1057

(52) U.S. Cl. CPC G16H 20/10; G06Q 10/1057

(57) ABSTRACT

A computer system may receive pharmacy information, where the pharmacy information specifies, for a potential prescription transaction for a prescription, a cost for an individual, an overhead associated with an entity when the potential prescription transaction is facilitated by the entity, and a residual amount. Then, the computer system may assess whether there is a prescription-transaction advantage when a second potential prescription transaction for the prescription is facilitated by the second entity. When there is the prescription-transaction advantage, the computer system may dynamically optimize or tailor a second cost for the individual and a second residual amount associated with the second potential prescription transaction. Next, when there is the prescription-transaction advantage, the computer system automatically and selectively performs the prescription transaction corresponding to the second potential prescription transaction with the second entity and using a second overhead associated with the second entity.

### CROSS REFERENCE TO RELATED APPLICATIONS

This application is a Continuation-in-Part of U.S. Non-Provisional application Ser. No. 17/398,915, “Dynamic Tailoring of a Prescription Transaction,” filed on Aug. 10, 2021, by Stanley Crane et al., the contents of which are herein incorporated by reference.

### FIELD

The described embodiments relate to techniques for dynamically tailoring a prescription transaction and providing an interactive comparison of prescription transactions when processed by different entities.

### BACKGROUND

Prescriptions for prescription drugs or medications are often filled or processed (which is sometimes referred to as a ‘prescription transaction’ or a ‘drug transaction’) by pharmacies. Many prescription transactions are facilitated by prescription benefits managers (PMBs). A PBM is typically a third-party administrator that manages a prescription drug program for a commercial health-insurance plan, a self-insured employer plan, a Medicare Part D plan, a Federal Employees Health Benefits program, a drug discount program, and/or a state government employee plan.

When a prescription transaction for a prescribed medication is facilitated by a PBM associated with an individual's health-insurance policy, the individual is typically charged a predefined copay or out-of-pocket fee specified by their health-insurance policy. Moreover, a pharmacy filling the individual's prescription during this prescription transaction usually charges an agreed drug ingredient cost for the prescribed medication as well as a margin for the pharmacy. Individuals may assume that the charges from the pharmacy exceed their copay (i.e., that their health insurer pays the difference as part of the health-insurance benefits), but for many prescriptions this is not the case. Instead, the charges from the pharmacy are often less than their copay. Consequently, when individuals fill many prescription facilitated by the PBM, the differences between the pharmacy charges and the copays (which are collected from the individuals by the pharmacy at the conclusion of the prescription transactions) are clawed back and paid from the pharmacy to the PBM and, thus, at least in part to the health insurer.

Furthermore, when an individual does not have health insurance, they may be forced to pay the full retail price for a prescription. However, the increased cost of the prescription may discourage the individual from filling the prescription or completing the prescription transaction. Such unfilled prescriptions increase the pharmacy workflow, result in increased operating costs, and can adversely impact the individual's health because of non-adherence to their prescribed therapy.

Alternatively, the individual may use a drug discount program (such as a discount card or a pharmacy application) when filling a prescription. In principle, drug discount programs can offer individuals reduced costs for at least some of their prescriptions. However, in practice, whether or not there is a reduced cost for a prescription with a drug discount program is often determined by running a trial claim. These trial claims increase the pharmacy workflow and operating costs (e.g., PBMs and health-insurance carriers typically charge a so-called ‘switch’ fee whenever a claim is submitted).

Additionally, as with many other aspects of current healthcare and health-insurance benefits, the details of many prescription transactions are typically opaque. Consequently, it is often difficult for individuals and pharmacies to get access to and, thus, to understand the details of prescription transactions. This makes it difficult for the individuals and the pharmacies to make informed decisions regarding the prescription transactions.

### SUMMARY

In a first group of embodiments, a computer system (which may include one or more computers, servers or electronic devices) that dynamically tailors a prescription transaction is described. This computer system may include: an interface circuit that communicates with a second computer system (which may include one or more computers, servers or electronic devices); a processor coupled to the interface circuit; and a memory, coupled to the processor, that stores program instructions. During operation, the computer system receives, at the interface circuit, pharmacy information associated with the second computer system, where the pharmacy information specifies, for a potential prescription transaction for a prescription, a cost for an individual, an overhead associated with an entity when the potential prescription transaction is facilitated by the entity, and a residual amount. Then, the computer system assesses whether there is a prescription-transaction advantage based at least in part on the overhead and a second overhead associated with a second entity when a second potential prescription transaction for the prescription is facilitated by the second entity. When there is the prescription-transaction advantage, the computer system dynamically optimizes, based at least in part on the prescription-transaction advantage and one or more prescription-transaction constraints, a second cost for the individual and a second residual amount associated with the second potential prescription transaction. Next, the computer system provides, from the interface circuit, comparison information associated with the second potential prescription transaction addressed to the second computer system, where the comparison information specifies the second cost and the second residual amount.

Note that the cost and the overhead may be predefined and may be associated with a PBM that manages prescription benefits.

Moreover, the pharmacy information may specify a potential prescription cost corresponding to a drug ingredient in the prescription and potential revenue for a pharmacy for the potential prescription transaction. Furthermore, the comparison information may specify second potential revenue for the pharmacy for the second potential prescription transaction. In some embodiments, the one or more prescription-transaction constraints may include that the second potential revenue is positive.

Additionally, the entity may be different from the pharmacy and may be different from the second entity.

Note that the entity may be a PBM or a provider of a prescription discount card for the prescription and the second entity may be a second PBM or a second provider of a second prescription discount card for the prescription.

Moreover, the second computer system may be associated with the pharmacy. For example, the pharmacy may have a contract with the second entity for a service that provides the comparison information.

Furthermore, the computer system may access historical information specifying prior prescription transactions for prescriptions of at least the individual, and the dynamic optimizing may be based at least in part on the historical information.

Additionally, the computer system may receive, at the interface circuit, at least one of the one or more prescription-transaction constraints associated with the second computer system. For example, the pharmacy information may specify at least one of the one or more prescription-transaction constraints.

In some embodiments, the computer system may: receive, at the interface circuit, acceptance information associated with the second computer system, where the acceptance information specifies that a prescription transaction for the prescription has been conducted between the pharmacy and the individual based at least in part on the comparison information; and selectively perform a second prescription transaction based at least in part on the acceptance information, where the second prescription transaction includes receiving the second overhead.

Note that the prescription-transaction advantage may correspond to a difference of the overhead and the second overhead, and there may be the prescription-transaction advantage when the overhead is greater than the second overhead.

Moreover, the comparison information may specify a comparison of the cost and the second cost, and/or a comparison of the residual amount and the second residual amount.

Furthermore, the one or more prescription-transaction constraints may be associated with the pharmacy.

Additionally, the dynamic optimization may segment the prescription-transaction advantage into a portion and a second portion, and the second cost may correspond to (or be a function of) a sum of the cost and the portion.

In some embodiments, the one or more prescription-transaction constraints may include a minimum potential revenue of the pharmacy. Note that the one or more prescription-transaction constraints may include: a preference for maximizing a potential revenue, a second preference for minimizing the second cost, or a third preference for apportioning a portion of the prescription-transaction advantage to the second cost and apportioning a second portion of the prescription-transaction advantage to the potential revenue.

Other embodiments provide the second computer system that performs counterpart operations to at least some of the aforementioned operations. Notably, the second computer system may include: a second interface circuit that communicates with the computer system; a second processor coupled to the second interface circuit; and a second memory, coupled to the second processor, that stores second program instructions. During operation, the second computer provides, from the second interface circuit, pharmacy information addressed to the computer system, where the pharmacy information specifies, for a potential prescription transaction for a prescription, a cost for an individual, an overhead associated with an entity when the potential prescription transaction is facilitated by the entity, and a residual amount. Then, the second computer system receives, at the interface circuit, comparison information associated with the computer system, where the comparison information specifies, for a second potential prescription transaction for the prescription that is facilitated by a second entity, a second cost for the individual and a second residual amount. Moreover, the second computer system provides, to a display, information specifying a user interface that includes the second comparison information with a comparison of the potential prescription transaction and the second potential prescription transaction, and the second comparison information includes the cost, the second code, the residual amount and the second residual amount. Next, the second computer system receives user-interface activity information indicating acceptance of the second potential prescription transaction. Furthermore, the second computer system selectively performs a prescription transaction for the prescription between the pharmacy and the individual based at least in part on the acceptance of the second potential prescription transaction.

Other embodiments provide a computer-readable storage medium with program instructions for use with the computer system or the second computer system. When executed by the computer system or the second computer system, the program instructions cause the computer system or the second computer system to perform at least some of the aforementioned operations in one or more of the preceding embodiments.

Other embodiments provide a method, which may be performed by the computer system or the second computer system. This method includes at least some of the aforementioned operations in one or more of the preceding embodiments.

In a second group of embodiments, a computer system (which may include one or more computers, servers or electronic devices) that automatically and selectively performs a prescription transaction is described. This computer system may include: an interface circuit; a processor coupled to the interface circuit; and a memory, coupled to the processor, that stores program instructions. During operation, the computer system receives pharmacy information, where the pharmacy information specifies, for a potential prescription transaction for a prescription, a cost for an individual, an overhead associated with an entity when the potential prescription transaction is facilitated by the entity, and a residual amount. Then, the computer system assesses whether there is a prescription-transaction advantage based at least in part on the overhead and a second overhead associated with a second entity when a second potential prescription transaction for the prescription is facilitated by the second entity. When there is the prescription-transaction advantage, the computer system dynamically optimizes, based at least in part on the prescription-transaction advantage and one or more prescription-transaction constraints, a second cost for the individual and a second residual amount associated with the second potential prescription transaction. Next, when there is the prescription-transaction advantage, the computer system automatically and selectively performs the prescription transaction corresponding to the second potential prescription transaction with the second entity and using the second overhead.

Note that the cost and the overhead may be predefined and may be associated with a PBM that manages prescription benefits.

Moreover, the pharmacy information may specify a potential prescription cost corresponding to a drug ingredient in the prescription and potential revenue for a pharmacy for the potential prescription transaction. Furthermore, the prescription transaction is associated with second potential revenue for the pharmacy. In some embodiments, the one or more prescription-transaction constraints may include that the second potential revenue is positive.

Additionally, the entity may be different from the pharmacy and may be different from the second entity.

Note that the entity may be a PBM or a provider of a prescription discount card for the prescription and the second entity may be a second PBM or a second provider of a second prescription discount card for the prescription.

Moreover, the computer system may be associated with the pharmacy. For example, the pharmacy may have a contract with the second entity for a service that determines when there is the prescription-transaction advantage.

Furthermore, the computer system may access historical information specifying prior prescription transactions for prescriptions of at least the individual, and the dynamic optimizing may be based at least in part on the historical information.

Additionally, the computer system may receive at least one of the one or more prescription-transaction constraints. For example, the pharmacy information may specify at least one of the one or more prescription-transaction constraints.

In some embodiments, the computer system may: receive acceptance information, where the acceptance information specifies that the prescription transaction for the prescription will be conducted between the pharmacy and the individual; and selectively performing of the prescription transaction is based at least in part on the acceptance information.

Note that the prescription-transaction advantage may correspond to a difference of the overhead and the second overhead, and there may be the prescription-transaction advantage when the overhead is greater than the second overhead.

Moreover, the performing the prescription transaction may include providing the prescription to the individual.

Furthermore, the one or more prescription-transaction constraints may be associated with the pharmacy.

Additionally, the dynamic optimization may segment the prescription-transaction advantage into a portion and a second portion, and the second cost may correspond to (or be a function of) a sum of the cost and the portion.

In some embodiments, the one or more prescription-transaction constraints may include a minimum potential revenue of the pharmacy. Note that the one or more prescription-transaction constraints may include: a preference for maximizing a potential revenue, a second preference for minimizing the second cost, or a third preference for apportioning a portion of the prescription-transaction advantage to the second cost and apportioning a second portion of the prescription-transaction advantage to the potential revenue.

Other embodiments provide a computer-readable storage medium with program instructions for use with the computer system. When executed by the computer system, the program instructions cause the computer system to perform at least some of the aforementioned operations in one or more of the preceding embodiments.

Other embodiments provide a method, which may be performed by the computer system. This method includes at least some of the aforementioned operations in one or more of the preceding embodiments.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of communication among electronic devices in accordance with an embodiment of the present disclosure.

FIG. 2 is a flow diagram illustrating an example of a method for providing comparison information using a computer system in FIG. 1 in accordance with an embodiment of the present disclosure.

FIG. 3 is a flow diagram illustrating an example of a method for receiving comparison information using a second computer system in FIG. 1 in accordance with an embodiment of the present disclosure.

FIG. 4 is a drawing illustrating an example of communication among an electronic device and computer systems in FIG. 1 in accordance with an embodiment of the present disclosure.

FIGS. 5-7 are drawings illustrating examples of comparisons of potential prescription transactions in accordance with an embodiment of the present disclosure.

FIG. 8 is a flow diagram illustrating an example of a method for providing comparison information using a computer system in FIG. 1 in accordance with an embodiment of the present disclosure.

FIG. 9 is a drawing illustrating an example of communication among an electronic device and computer systems in FIG. 1 in accordance with an embodiment of the present disclosure.

FIG. 10 is a block diagram illustrating an example of an electronic device in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

During operation, a computer system may receive pharmacy information that specifies, for a potential prescription transaction for a prescription, a cost for an individual, an overhead associated with an entity when the potential prescription transaction is facilitated by the entity, and a residual amount. Then, the computer system may assess whether there is a prescription-transaction advantage based at least in part on the overhead and a second overhead associated with a second entity when a second potential prescription transaction for the prescription is facilitated by the second entity. When there is the prescription-transaction advantage, the computer system may dynamically optimize or tailor, based at least in part on the prescription-transaction advantage and one or more prescription-transaction constraints, a second cost for the individual and a second residual amount associated with the second potential prescription transaction. Next, when there is the prescription-transaction advantage, the computer system automatically and selectively performs the prescription transaction corresponding to the second potential prescription transaction with the second entity and using the second overhead.

By selectively dynamically tailoring the second potential prescription transaction and selectively performing the prescription transaction, these prescription techniques may provide improved service. Notably, the prescription techniques may create the second potential prescription transaction for the benefit of the individual and/or a pharmacy. For example, depending on the one or more prescription-transaction constraints (which may, at least in part, be specified by the pharmacy), the prescription techniques may optimize the second potential prescription transaction for savings of the individual (such as a reduced second cost) and/or revenue of the pharmacy (such as an increased second residual amount). In the process, the prescription techniques may: reduce unfilled prescriptions (thereby reducing pharmacy workflow and operating costs, while potentially improving the health of individuals), reduce the use of trial claims (thereby reducing pharmacy workflow and operating costs), and/or provide improved transparency (thereby allowing individuals and pharmacies to make more informed decisions regarding prescription transactions and, thus, provide the individuals and the pharmacies improved pricing power). Moreover, reducing or eliminating the use of trial claims, the prescription techniques may improve the function of the computer system by reducing the use of resources, such as: network bandwidth, processor cycles, and memory. Consequently, the dynamic pricing in the prescription techniques may be used to: increase customer satisfaction (and, thus, loyalty), increase profitability of the pharmacy, improve market efficiency, and/or provide a more equitable market.

In the discussion that follows, electronic devices, computers and/or servers (which may be local or remotely located from each other) may communicate packets in accordance with a wired communication protocol and/or a wireless communication protocol. The wireless communication protocol may include: a wireless communication protocol that is compatible with an Institute of Electrical and Electronics Engineers (IEEE) 802.11 standard (which is sometimes referred to as ‘Wi-Fi®,’ from the Wi-Fi Alliance of Austin, Texas), Bluetooth, a cellular-telephone network or data network communication protocol (such as a third generation or 3G communication protocol, a fourth generation or 4G communication protocol, e.g., Long Term Evolution or LTE (from the 3rd Generation Partnership Project of Sophia Antipolis, Valbonne, France), LTE Advanced or LTE-A, a fifth generation or 5G communication protocol, or other present or future developed advanced cellular communication protocol), and/or another type of wireless interface (such as another wireless-local-area-network interface). For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies. Moreover, the wired communication protocol may include a wired communication protocol that is compatible with an IEEE 802.3 standard (which is sometimes referred to as ‘Ethernet’), e.g., an Ethernet II standard. However, a wide variety of communication protocols may be used. In the discussion that follows, Wi-Fi, LTE and Ethernet are used as illustrative examples.

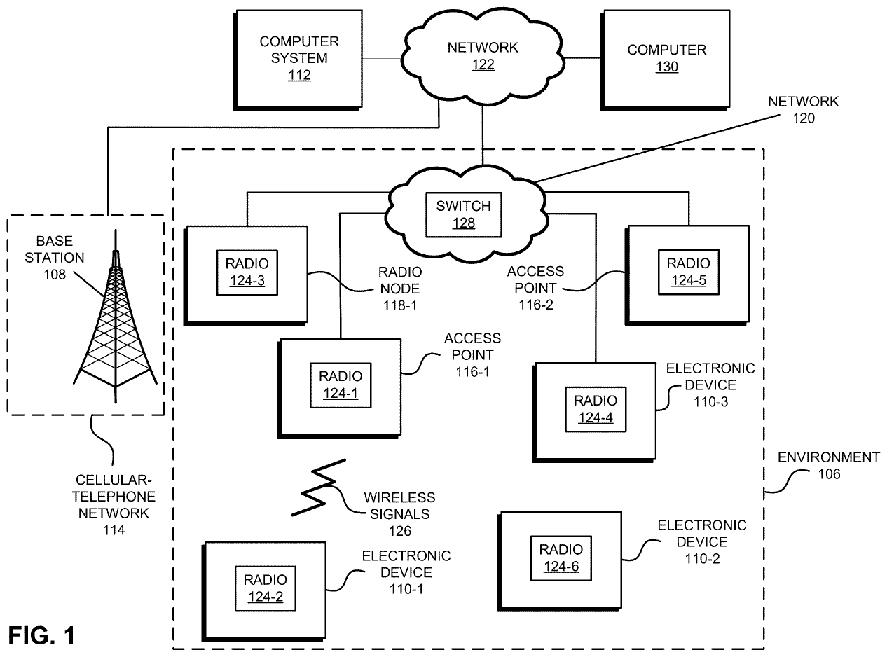

We now describe some embodiments of the prescription techniques. FIG. 1 presents a block diagram illustrating an example of communication in an environment 106 with one or more electronic devices 110 (such as cellular telephones, portable electronic devices, stations or clients, another type of electronic device, etc.) via a cellular-telephone network 114 (which may include a base station 108), one or more access points 116 (which may communicate using Wi-Fi) in a wireless local area network (WLAN) and/or radio node 118 (which may communicate using LTE) in a small-scale network (such as a small cell). For example, radio node 118 may include: an Evolved Node B (eNodeB), a Universal Mobile Telecommunications System (UMTS) NodeB and radio network controller (RNC), a New Radio (NR) gNB or gNodeB (which communicates with a network with a cellular-telephone communication protocol that is other than LTE), etc. In the discussion that follows, an access point, a radio node or a base station are sometimes referred to generically as a ‘communication device.’ Moreover, one or more base stations (such as base station 108), access points 116, and/or radio node 118 may be included in one or more networks, such as: a WLAN, a small cell, a local area network (LAN) and/or a cellular-telephone network. In some embodiments, access points 116 may include a physical access point and/or a virtual access point that is implemented in software in an environment of an electronic device or a computer.

Note that access points 116 and/or radio node 118 may communicate with each other and/or computer system 112 (which may include one or more computers or servers, and which may be implemented locally or remotely to provide storage and/or back-office services for one or more pharmacies) using a wired communication protocol (such as Ethernet) via network 120 and/or 122. Note that networks 120 and 122 may be the same or different networks. For example, networks 120 and/or 122 may be a LAN, an intra-net or the Internet. Consequently, in some embodiments, the wired communication protocol may include a secured connection over transmission control protocol/Internet protocol (TCP/IP) using hypertext transfer protocol secure (HTTPS) with a JavaScript object notation (JSON) Web services connection. In some embodiments, network 120 may include one or more routers and/or switches (such as switch 128).

Moreover, computer system 112 may communicate with computer system 130 (which may include one or more computers or servers, and which may be implemented locally or remotely) using a wired communication protocol (such as Ethernet) via network 122. Computer system 130 may implement at least some of the operations in the prescription techniques. Notably, as described further below, computer system 130 may dynamically optimize or tailor one or more pharmacy transactions.

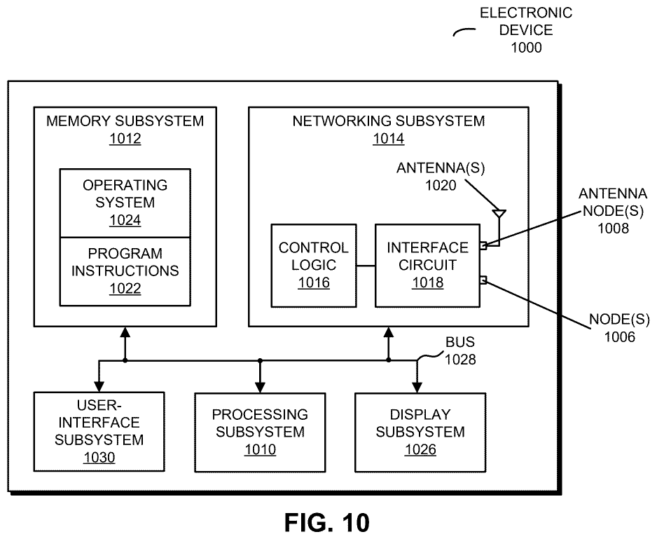

As described further below with reference to FIG. 10, base station 108, electronic devices 110, computer system 112, access points 116, radio node 118, switch 128 and/or computer system 130 may include subsystems, such as a networking subsystem, a memory subsystem and a processor subsystem. In addition, electronic devices 110, access points 116 and radio node 118 may include radios 124 in the networking subsystems. More generally, electronic devices 110, access points 116 and radio node 118 can include (or can be included within) any electronic devices with the networking subsystems that enable electronic devices 110, access points 116 and radio node 118 to wirelessly communicate with one or more other electronic devices. This wireless communication can comprise transmitting access on wireless channels to enable electronic devices to make initial contact with or detect each other, followed by exchanging subsequent data/management frames (such as connection requests and responses) to establish a connection, configure security options, transmit and receive frames or packets via the connection, etc.

During the communication in FIG. 1, base station 108, electronic devices 110, computer system 112, access points 116, radio node 118 and/or computer system 130 may wired or wirelessly communicate while: transmitting access requests and receiving access responses on wired or wireless channels, detecting one another by scanning wireless channels, establishing connections (for example, by transmitting connection requests and receiving connection responses), and/or transmitting and receiving frames or packets (which may include information as payloads).

As can be seen in FIG. 1, wireless signals 126 (represented by a jagged line) may be transmitted by radios 124 in, e.g., access points 116 and/or radio node 118 and electronic devices 110. For example, radio 124-1 in access point 116-1 may transmit information (such as one or more packets or frames) using wireless signals 126. These wireless signals are received by radios 124 in one or more other electronic devices (such as radio 124-2 in electronic device 110-1). This may allow access point 116-1 to communicate information to other access points 116 and/or electronic device 110-1. Note that wireless signals 126 may convey one or more packets or frames.

In the described embodiments, processing a packet or a frame in one or more electronic devices in electronic devices 110, computer system 112, access points 116, radio node 118 and/or computer system 130 may include: receiving the wireless or electrical signals with the packet or the frame; decoding/extracting the packet or the frame from the received wireless or electrical signals to acquire the packet or the frame; and processing the packet or the frame to determine information contained in the payload of the packet or the frame.

Note that the wired and/or wireless communication in FIG. 1 may be characterized by a variety of performance metrics, such as: a data rate for successful communication (which is sometimes referred to as ‘throughput’), an error rate (such as a retry or resend rate), a mean-squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). While instances of radios 124 are shown in components in FIG. 1, one or more of these instances may be different from the other instances of radios 124.

In some embodiments, wireless communication between components in FIG. 1 uses one or more bands of frequencies, such as: 900 MHz, 2.4 GHz, 5 GHz, 6 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. Note that the communication between electronic devices may use multi-user transmission (such as orthogonal frequency division multiple access or OFDMA).

Although we describe the network environment shown in FIG. 1 as an example, in alternative embodiments, different numbers or types of electronic devices may be present. For example, some embodiments comprise more or fewer electronic devices. As another example, in another embodiment, different electronic devices are transmitting and/or receiving packets or frames.

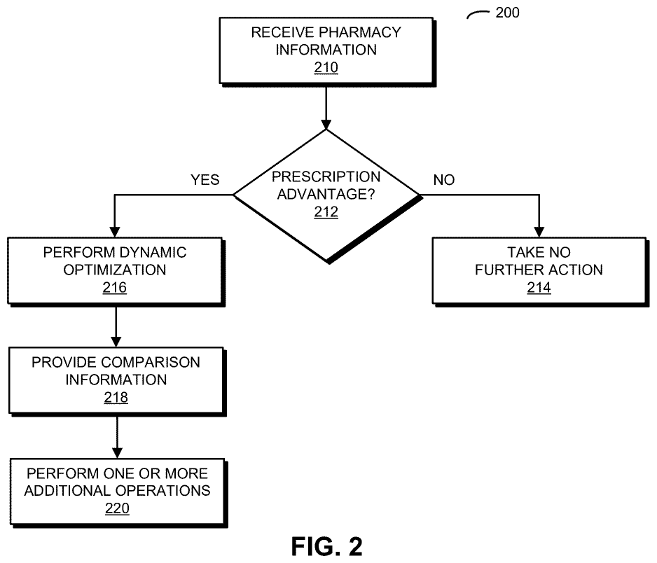

As discussed previously, existing approaches for conduction prescription transactions may be: opaque, inefficient and/or unequitable. In order to address these problems, as described further below with reference to FIGS. 2-7, embodiments of the prescription techniques may be used to dynamically tailor or optimize one or more prescription transactions. Notably, the prescription techniques may be performed when an individual is filling a prescription or conducting a prescription transaction at or with a pharmacy. For example, electronic device 110-3 (such as a point-of-sale terminal or cash register) in the pharmacy may a notification or a message to computer system 112 (e.g., via an application programming interface or API). In response, computer system 112 may provide pharmacy information to computer system 130 (e.g., via another API), where the pharmacy information specifies, for a potential prescription transaction for a prescription, a cost for an individual, an overhead associated with an entity when the potential prescription transaction is facilitated by the entity, and a residual amount. Note that the cost and the overhead may be predefined and may be associated with a PBM that manages prescription benefits, such as a for a health-insurance provider of the individual.

In some embodiments, computer system 112 optionally provides one or more prescription-transaction constraints to computer system 130. The one or more prescription-transaction constraints may be associated with the pharmacy. Note that the one or more prescription-transaction constraints may be provided in advance of the individual filling the prescription at the pharmacy. Alternatively, the one or more prescription-transaction constraints may be provided while the individual is filling the prescription or conducting the prescription transaction. In these embodiments, the one or more prescription-transaction constraints may be dynamically determined by computer system 112, such as based at least in part on historical information about previous prescription transactions of at least the individual. For example, if the individual is considered likely to switch to a different pharmacy, the one or more prescription-transaction constraints may specify that the dynamic optimizing described further below should reduce or minimize the cost to the individual. Alternatively, if the individual is considered to be a stable customer, the one or more prescription-transaction constraints may specify that the dynamic optimizing should increase or maximize the revenue of the pharmacy.

After receiving the pharmacy information and/or the one or more prescription-transaction constraints, computer system 130 may assess whether there is a prescription-transaction advantage based at least in part on the overhead and a second overhead associated with a second entity when a second potential prescription transaction for the prescription is facilitated by the second entity. For example, there may be the prescription-transaction advantage when the overhead is greater than the second overhead (and, thus, when the second entity is able to or willing to accept a smaller amount in the second potential prescription transaction than the entity is able to or willing to accept in the potential prescription transaction).

When there is the prescription-transaction advantage, computer system 130 may dynamically optimize, based at least in part on the prescription-transaction advantage and one or more prescription-transaction constraints, a second cost for the individual and a second residual amount associated with the second potential prescription transaction. For example, the dynamic optimization may dynamically price the second potential prescription transaction based at least in part on the revenue of the pharmacy and a cost to the individual.

Next, computer system 130 may provide comparison information associated with the second potential prescription transaction to computer system 112, where the comparison information specifies the second cost and the second residual amount. After receiving the comparison information, computer system 112 may provide the comparison information to electronic device 110-3. Moreover, after receiving the comparison information, electronic device 110-3 may provide the comparison information to the individual and/or a staff member or employee in the pharmacy. For example, the comparison information may be displayed on a display associated with a point-of-sale terminal. Notably, electronic device 110-3 may display the comparison information in a user interface that includes a comparison of the potential prescription transaction and the second potential prescription transaction, and the second comparison information includes, e.g., the cost, the second code, the residual amount and the second residual amount.

When the second potential prescription transaction is less costly for the individual and/or offers more revenue for the pharmacy, the individual or the staff member in the pharmacy may select the potential prescription transaction or the second potential prescription transaction. For example, the individual or the staff member may, directly or indirectly (e.g., via a human-interface device, such as a keyboard, a mouse, a touchpad, a stylus, a touch-sensitive display, a voice-recognition interface, etc.), provide user-interface activity information indicating acceptance of the second potential prescription transaction. In response, electronic device 110-3 may provide a notification to computer system 112.

Then, the staff member may conduct the prescription transaction with the individual. This may include electronic device 110-3 collecting or receiving payment from the individual (including the second cost). Furthermore, electronic device 110-3 may notify computer system 112 that the second potential transaction was accepted so that computer system 112 can update its records. Additionally, computer system 112 may notify computer system 130 that the second potential transaction was accepted.

In this way, the prescription techniques may to provide improved service, such as reduced cost for individuals and/or increased profitability for pharmacies. Notably, by dynamically optimizing or tailoring the second prescription transaction and providing the comparison information, the prescription techniques may offer individuals and/or the pharmacies transparent, real-time information that facilitates improved decision-making as to how best to conduct prescription transactions that are in their best interests.

While the preceding discussion illustrated the use of the prescription techniques when a prescription transaction is conducted in-person (e.g., at the pharmacy), in other embodiments the prescription techniques may be used during an online prescription transaction, e.g., when the individual is filling their prescription with the pharmacy virtually via networks 120 and/or 122.

We now describe embodiments of the method. FIG. 2 presents a flow diagram illustrating an example of a method 200 for providing comparison information, which may be performed by a computer system, such as computer system 130 in FIG. 1. During operation, the computer system may receive pharmacy information (operation 210) associated with the second computer system, where the pharmacy information specifies, for a potential prescription transaction for a prescription, a cost for an individual, an overhead associated with an entity when the potential prescription transaction is facilitated by the entity, and a residual amount.

Note that the cost and the overhead may be predefined and may be associated with a PBM that manages prescription benefits. Moreover, the pharmacy information may specify a potential prescription cost corresponding to a drug ingredient in the prescription and potential revenue for a pharmacy for the potential prescription transaction.

Then, the computer system may assess whether there is a prescription-transaction advantage (operation 212) based at least in part on the overhead and a second overhead associated with a second entity when a second potential prescription transaction for the prescription is facilitated by the second entity. When there is no prescription-transaction advantage (operation 212), the computer system may take no further action (operation 214). Alternatively, when there is the prescription-transaction advantage (operation 212), the computer system may perform dynamically optimization (operation 216). For example, the dynamic optimization, based at least in part on the prescription-transaction advantage and one or more prescription-transaction constraints, may determine a second cost for the individual and a second residual amount associated with the second potential prescription transaction.

Note that the entity may be different from the pharmacy and may be different from the second entity. For example, the entity may be a PBM or a provider of a prescription discount card for the prescription and the second entity may be a second PBM or a second provider of a second prescription discount card for the prescription.

Moreover, the second computer system may be associated with the pharmacy. For example, the pharmacy may have a contract with the second entity for a service that provides the comparison information.

Furthermore, the prescription-transaction advantage may correspond to or may be a function of a difference of the overhead and the second overhead, and there may be the prescription-transaction advantage when the overhead is greater than the second overhead (and, thus, when the second entity is able to or willing to accept a smaller amount in the second potential prescription transaction than the entity is able to or willing to accept in the potential prescription transaction).

Additionally, the one or more prescription-transaction constraints may be associated with the pharmacy. For example, the pharmacy (or a corporation that owns multiple pharmacies, which include the pharmacy) may provide or specify the one or more prescription-transaction constraints. In some embodiments, the one or more prescription-transaction constraints may be dynamically varied by the pharmacy, such as from one individual to another or for the individual as a function of time (such as based at least in part on the historical information described further below).

The dynamic optimization may segment the prescription-transaction advantage into a portion and a second portion, and the second cost may correspond to (or be a function of) a sum of the cost and the portion. In some embodiments, the one or more prescription-transaction constraints may include a minimum potential revenue of a pharmacy. Alternatively or additionally, the one or more prescription-transaction constraints may include: a preference for maximizing a potential revenue, a second preference for minimizing the second cost, or a third preference for apportioning a portion of the prescription-transaction advantage to the second cost and apportioning a second portion of the prescription-transaction advantage to the potential revenue. Thus, depending on the preferences of the pharmacy (as specified by the one or more prescription-transaction constraints), the computer system may dynamically optimize or tailor the second potential prescription transaction to the advantage of the pharmacy, the advantage of the individual or any dynamic point in between.

Next, the computer system may provide the comparison information (operation 218) associated with the second potential prescription transaction addressed to the second computer system, where the comparison information specifies the second cost and the second residual amount. Note that the comparison information may specify second potential revenue for the pharmacy for the second potential prescription transaction. In some embodiments, the one or more prescription-transaction constraints may include that the second potential revenue is positive. Furthermore, the comparison information may specify a comparison of the cost and the second cost, and/or a comparison of the residual amount and the second residual amount.

In some embodiments, the computer system may optionally perform one or more additional operations (operation 220). For example, the computer system may access historical information specifying prior prescription transactions for prescriptions of at least the individual, and the dynamic optimizing may be based at least in part on the historical information.

Moreover, the computer system may receive at least one of the one or more prescription-transaction constraints associated with the second computer system. For example, the pharmacy information may specify at least one of the one or more prescription-transaction constraints.

Furthermore, the computer system may: receive acceptance information associated with the second computer system, where the acceptance information specifies that a prescription transaction for the prescription has been conducted between the pharmacy and the individual based at least in part on the comparison information; and selectively perform a second prescription transaction based at least in part on the acceptance information, where the second prescription transaction includes receiving the second overhead.

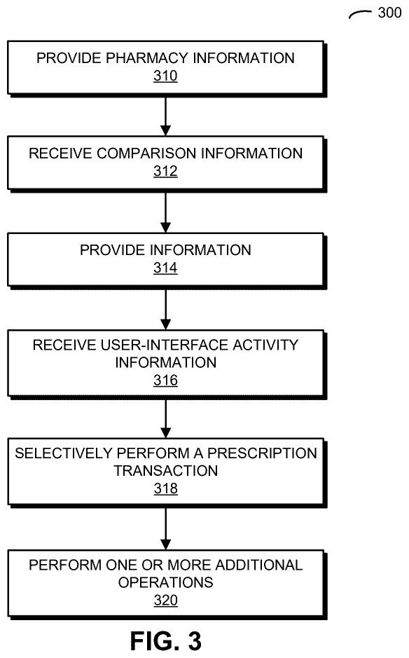

FIG. 3 presents a flow diagram illustrating an example of a method 300 for receiving comparison information, which may be performed by a second computer system, such as electronic device 110-3 or computer system 112 in FIG. 1. During operation, the second computer system may provide pharmacy information (operation 310) addressed to the computer system, where the pharmacy information specifies, for a potential prescription transaction for a prescription, a cost for an individual, an overhead associated with an entity when the potential prescription transaction is facilitated by the entity, and a residual amount. Then, the second computer system may receive the comparison information (operation 312) associated with (or from) the computer system, where the comparison information specifies, for a second potential prescription transaction for the prescription that is facilitated by a second entity, a second cost for the individual and a second residual amount.

Moreover, the second computer system may provide, to a display, information (operation 314) specifying a user interface that includes the second comparison information with a comparison of the potential prescription transaction and the second potential prescription transaction, and the second comparison information includes the cost, the second code, the residual amount and the second residual amount. For example, the display may be included in or proximate to the second computer system, such as in embodiments where the second computer system is included in the pharmacy (e.g., in or proximate to a point-of-sale terminal). Alternatively, the display may be remotely located from the second computer system, such as in embodiments where the display is included in or proximate to a point-of-sale terminal in the pharmacy and the second computer system is remotely located. Thus, at least some of the operations in the prescription techniques may be implemented in a central and/or a distributed manner.

Next, the second computer system may receive user-interface activity information (operation 316) indicating acceptance of the second potential prescription transaction. For example, the second computer system may include a user-interface device (such as a keyboard, a mouse, a trackpad, a stylus, a touch-sensitive display, a voice-recognition interface, etc.) that receives the user-interface activity information from the individual or a sales associate at the pharmacy. Alternatively, the second computer system may receive the user-interface activity information from a point-of-sale terminal in the pharmacy.

Furthermore, the second computer system may selectively perform a prescription transaction (operation 318) for the prescription between the pharmacy and the individual based at least in part on the acceptance of the second potential prescription transaction. For example, the prescription transaction may include the second cost, the second overhead and/or the second residual amount associated with the second potential prescription.

In some embodiments, the second computer system may optionally perform one or more additional operations (operation 320). For example, when the second computer system selectively performs the prescription transaction, the second computer system may provide the second overhead to the computer system. Notably, the second computer may provide instructions to conduct an electronic payment of the second overhead to a financial account associated with a provider of the computer system (such as at the end of business day) and/or may otherwise credit the provider of the computer system for the second overhead.

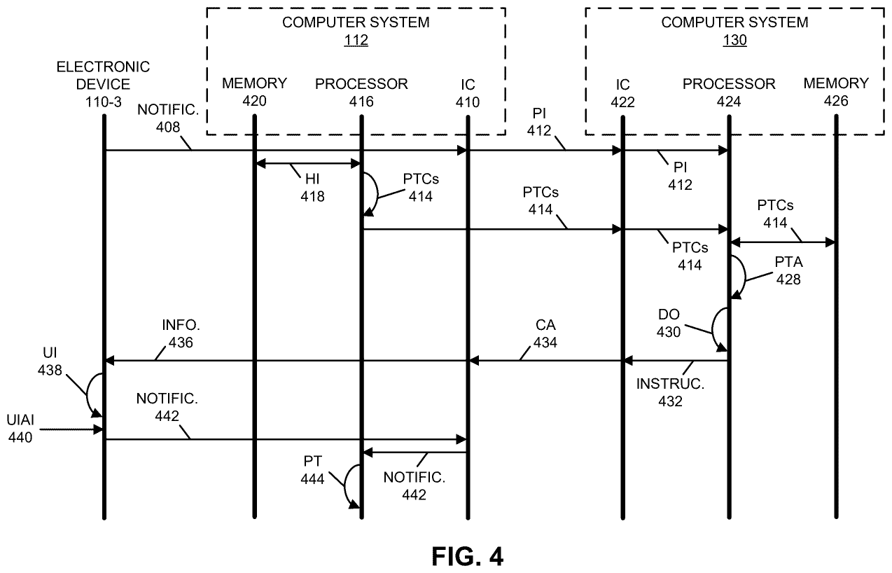

Embodiments of the prescription techniques are further illustrated in FIG. 4, which presents a drawing illustrating an example of communication among electronic device 110-3, computer system 112 and computer system 130. In FIG. 4, an interface circuit (IC) 410 in computer system 112 may provide pharmacy information (PI) 412 to computer system 130, where the pharmacy information specifies, for a potential prescription transaction for a prescription, a cost for an individual, an overhead associated with an entity when the potential prescription transaction is facilitated by the entity, and a residual amount. For example, computer system 112 may provide pharmacy information 412 when the individual is at a point-of-sale terminal in a pharmacy associated with computer system 112, such as when the individual is filling or picking up the prescription at the pharmacy (and, thus, when the individual is conducting or is about to conduct a prescription transaction). Notably, computer system 112 may provide pharmacy information 412 based at least in part on a notification 408 received from electronic device 110-3, such as the point-of-sale terminal in the pharmacy. This notification may include information that specifies a prescription drug benefit associated with the individual (such as a health-insurance policy, an associated PBM, etc.) and a prescription associated with the individual. Alternatively, in some embodiments, computer system 112 may provide pharmacy information 412 in advance, such as after a prescription is submitted to the pharmacy but before the individual picks up the prescription at or from the pharmacy.

In some embodiments, interface circuit 410 optionally provides one or more prescription-transaction constraints (PTCs) 414 to computer system 130. The one or more prescription-transaction constraints 414 may be associated with the pharmacy. Note that the one or more prescription-transaction constraints 414 may be provided in advance of the individual filling the prescription at the pharmacy. Alternatively, the one or more prescription-transaction constraints 414 may be provided while the individual is filling the prescription or conducting the prescription transaction. In these embodiments, the one or more prescription-transaction constraints 414 may be dynamically determined by a processor 416 in computer system 112, such as based at least in part on historical information (HI) 418 about previous prescription transactions of at least the individual, which may be stored in memory 420 in or associated with computer system 112.

After receiving pharmacy information 412 and/or the one or more prescription-transaction constraints 414, interface circuit 422 in computer system 130 may provide this information to processor 424 in computer system 130. Alternatively or additionally, processor 424 may access at least one of the one or more prescription-transaction constraints 414 in memory 426 in computer system 130.

Then, processor 424 may assess whether there is a prescription-transaction advantage (PTA) 428 based at least in part on the overhead and a second overhead associated with a second entity when a second potential prescription transaction for the prescription is facilitated by the second entity. When there is the prescription-transaction advantage 428, processor 424 may dynamically optimize (DO) 430, based at least in part on the prescription-transaction advantage and one or more prescription-transaction constraints, a second cost for the individual and a second residual amount associated with the second potential prescription transaction. In general, dynamic optimization 430 may be over one or more variables. For example, dynamic optimization 430 may attempt to maximize a revenue of the pharmacy, while minimizing a cost to the individual.

Next, processor 424 may instruct 432 interface circuit 422 to provide comparison information (CA) 434 associated with the second potential prescription transaction to computer system 112, where comparison information 434 specifies the second cost and the second residual amount. After receiving the comparison information 434, interface circuit 434 may provide information 436 (which includes comparison information 434) to electronic device 110-3.

After receiving information 436, electronic device 110-3 may display second comparison information of different potential prescription transactions. For example, electronic device 110-3 may display the second comparison information in a user interface 438 that includes a comparison of the potential prescription transaction and the second potential prescription transaction, and the second comparison information includes the cost, the second code, the residual amount and the second residual amount.

Based at least in part on the displayed second comparison information (such as when the second potential prescription transaction is less costly for the individual and/or offers more revenue for the pharmacy), the individual and/or a staff member in the pharmacy may select the potential prescription transaction or the second potential prescription transaction. When electronic device receives user-interface activity information (UIAI) 440 indicating acceptance of the second potential prescription transaction, electronic device 110-3 may provide a notification 442 to computer system 112.

In response to receiving UIAI 440, interface circuit 410 provide notification 442 to processor 424. Then, processor 424 may selectively perform a prescription transaction (PT) 444 for the prescription between the pharmacy and the individual based at least in part on the acceptance of the second potential prescription transaction. Note that selectively performing prescription transaction 444 may include: notifying computer system 130 about prescription transaction 444; and/or instructing electronic device 110-3 to provide an instruction to staff at the pharmacy to collect the payment from the individual (including the second cost).

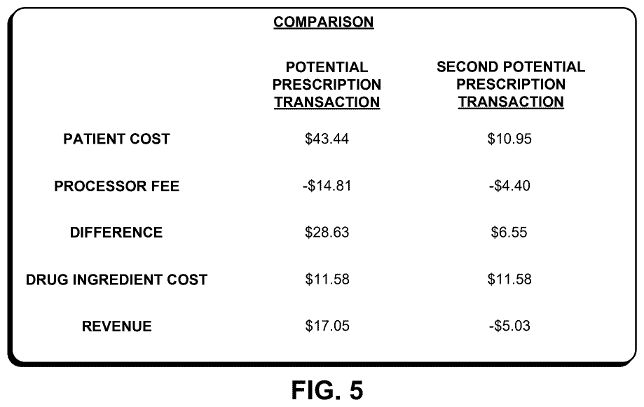

We now describe embodiments of potential prescription transactions and comparison information. FIGS. 5-7 present drawings illustrating examples of comparisons of potential prescription transactions. Notably, FIG. 5 illustrates an example in which the second potential prescription transaction is dynamically optimized to minimize the cost to the individual. In particular, the second potential prescription transaction may offer a saving of $32.49 for the patient relative to the potential prescription transaction (i.e., the cost associated with their current health-insurance plan).

However, in this example, this may cause the pharmacy to lose money ($5.03) on this prescription transaction. In general, most pharmacists, pharmacy chains, and independent pharmacy owners are typically narrowly focused on profitability, which may result in the individual paying a non-competitive price so that higher profit margins may be realized. In the example in FIG. 5, the pharmacist may choose to process the prescription transaction or claim under the current price (i.e., according to the potential prescription transaction) versus the lowest price for the individual (i.e., according to the second potential prescription transaction) in order to have a profit of $17.05 instead of a loss of $5.03 on the prescription transaction.

Note that in FIG. 5, the processor fee may be the overhead associated with the given entity, the patient cost may be the given cost of the individual, the difference may be the residual amount, the drug ingredient cost may be a given prescription cost corresponding to a drug ingredient in the prescription and revenue may be given potential revenue for the pharmacy.

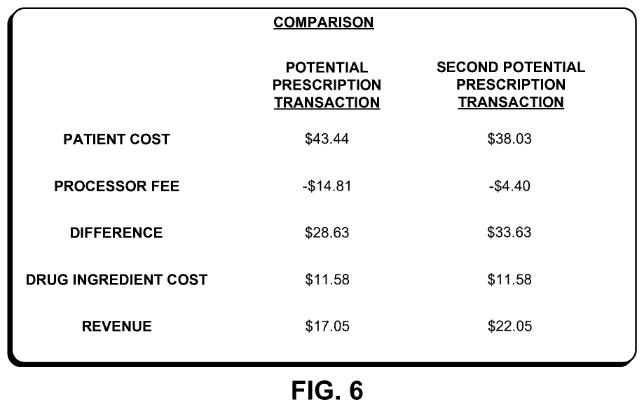

Alternatively, as shown in FIG. 6, which presents a drawing illustrating an example of a comparison of potential prescription transactions, the prescription techniques may provide real-time dynamic pricing to enhance the revenue of the pharmacy. In particular, in current market conditions, the pharmacy may be challenged to decide whether to focus on ingredient margin and profitability, or to deliver the lowest cost to the individual (who may be a loyal customer). For example, drug discount card programs and/or health-insurance copays may contain excessive administrative fees and ingredient spreads (the difference between the drug acquisition cost and the price a PBM charges a health-insurance carrier for a drug), which often cause the dispensing pharmacy to lose money on a prescription transaction of a claim, while the discount programs and health-insurance carriers realize significant profits and claw backs. The disclosed prescription techniques may level the playing field by dynamically repricing and adjudicating claims in real-time to increase or maximize pharmacy margins and/or deliver reduce or minimal cost to the individual. In the example shown in FIG. 6, the second potential prescription transaction has a lower cost for the individual and increased revenue for the pharmacy relative to the potential prescription transaction associated with the current health-insurance plan of the individual.

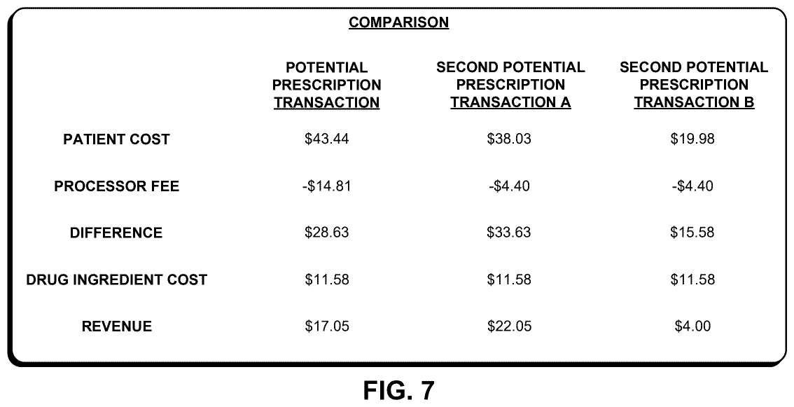

Another embodiment is shown in FIG. 7, which presents a drawing illustrating an example of a comparison of potential prescription transactions. In FIG. 7, the potential prescription transaction associated with the current health-insurance plan has a large processor fee, administration fee and/or claw back of $14.81. The use of real-time dynamic pricing provides the individual and/or the pharmacy with at least two pricing options to choose from for a particular prescription transaction. If the pharmacy focus is on revenue or profitability, second potential prescription transaction A may be selected. This increases the revenue of the pharmacy to $22.05, but also reduces the cost to the individual to $38.03. However, if the pharmacy focus is on reduced cost for the individual, second potential prescription transaction B may be selected (e.g., by the pharmacy). This further reduces the cost to the individual to $19.98, and reduces the pharmacy revenue to $4.00. Note that regardless of which of the second potential prescription transactions is selected, the pharmacy may not have a loss on the prescription transaction.

Note that the comparisons shown in FIGS. 5-7 may include less information, more information, and/or different information. For example, information in two or more items may be combined into or represented by a single item, information in a single item may be separated into or represented by two or more items, and/or information in a given item may be removed or replaced by information in another item.

We now describe the dynamic optimization performed by the computer system during the prescription techniques. Note that medications may be grouped by one or more difference techniques into groups or classes, e.g., based at least in part on characteristics or attributes of or associated with the medications. In the prescription techniques, a given grouping may be used to derive a dynamic price.

The following series may include one or more data-structure or database transactions. AN_{iq} may represent, for a quantity q in a series of prescription transactions i, the amount an insurance company, drug discount card or a pharmacy management system (PMS) would instruct a pharmacy to charge an individual for a prescription for medication of any national drug code (NDC) in a particular group or class. Moreover, FN_{iq} may represent, for a quantity q in a series of prescription transactions i, the amount an insurance company, drug discount card or PMS would fund or charge the pharmacy to fill a prescription with medication of any NDC in a particular group or class. Furthermore, CN_{iq} may represent, for a quantity q in a series of prescription transactions i, the cost to a pharmacy for a medication of any NDC in a particular group or class. Additionally, QN_{iq} may represent the metric quantity of the medication in a prescription in a series of prescription transactions i. In some embodiments, WN_{q} may represent, for a quantity q in a series of prescription transactions i, the dynamic price in the disclosed prescription techniques that the pharmacy would collect from the individual for a prescription for medication of any NDC in a particular group or class. Note that: XN may represent, for a quantity q in a series of prescription transactions i, the pharmacy dispensing fee in the disclosed prescription techniques paid for a prescription for medication with NDC N; YN may represent, for a quantity q in a series of prescription transactions i, the charge the disclosed prescription techniques would require to fill a prescription for medication with NDC N; and ZN may represent the percentage of the difference applied to the individual.

If YN is greater than FN_{iq}, then WN_{q} may be determined or computed from the value of W that maximizes this count, over all values of i and quantities q. Notably,

$$W \cdot {QN}_{iq} + XN + YN \leq {AN}_{i q} and W \cdot {QN}_{iq} - YN + XN \geq {AN}_{i q} - {FN}_{iq} , so that {WN}_{q} = W \cdot {QN}_{iq} + XN + YN . \qquad ( 1 )$$

Moreover, if YN is less than or equal to FN_{iq}, then

$$W {N}_{q} = A {N}_{iq} + ( YN - {FN}_{iq} ) \cdot ZN . \qquad ( 2 )$$

Note that AN_{iq} may range from a few dollars to hundreds of thousands of dollars. However, the average of AN_{iq} may be between $40 and $60 for current medication prices. Moreover, FN_{iq} may range from $0.25 to $300, and may have an average value between $8 and $12. Furthermore, CN_{iq} may be $1 to $2 below AN_{iq}. However, in some embodiments, CN_{iq} may be higher than AN_{iq}, which may result in the pharmacy losing money when filling or fulfilling that prescription. Additionally, WN_{q} may be less than AN_{iq} by $1 to $300, with an averaging value between $35 and $55. Note that XN may be between $1 and $3, and YN may vary dynamically. For example, YN may be between $0.027 and $6.00 (or even a larger value).

In some embodiments where YN is less than or equal to FN_{iq}, then WN_{q} may be determined or computed using Eqn. 2. For example, if YN equals $3, FN_{iq} equals $22.49, AN_{iq} equals $30.53 and ZN equals 0.50, then WN_{q}=$30.53+($3−$22.49)·0.50=$20.78.

Moreover, if YN is greater than FN_{iq} then WN_{q} may be determined or computed using Eqn. 1. We consider several examples in the following discussion.

Notably, in a cross-medication-iteration approach, for every medication N, an initial ingredient fee, normalized to quantity of one (which may be represented as VN₁), may be calculated from historical data based at least in part on one or more factors (such as the mean ingredient fee, median ingredient fee, and/or other historical values). Furthermore, a range of ingredient fees (which is represented as RN) may be calculated based at least in part on one or more factors (such as a standard deviation of normalized ingredient reimbursement). Then, a first iteration of WN₁, WN₁₋₁, may be calculated based at least in part on VN₁ plus YN plus XN. This may be performed for all medications, of which there may be, e.g., more than 50,000. Note that the adjudication rate for WN₁₋₁ may be measured for a trial period, after which each WN₁₋₁ may be adjusted based at least in part on the RN of this medication, and WN₁₋₂ (a second iteration of WN₁) may be derived for every medication as a positive or negative percentage of each the RN for each medication. In some embodiments, the adjudication rate for WN₁₋₂ may be measured for another trial period and, based at least in part on the increase or decrease of the adjudication rates across all medications and all WN₁₋₁ and WN₁₋₂, WN₁₋₃ (a third iteration of WN₁) may be derived using the knowledge gained from the changing adjudication rates for all medications. For example, if WN₁₋₁ is $10.00 and has an adjudication rate of 10%, and WN₁₋₂ is $11.00 and had an adjudication rate of 12%, then WN₁₋₃ may be larger than $11.00. Moreover, based at least in part on the WN₁₋₃ adjudication rate, WN₁₋₄ (a fourth iteration of WN₁) may reflect the knowledge of the previous iterations of WN₁ and their respective adjudication rates.

Note that the trail period may be between a few days to a few weeks. Alternatively, the trial period may be: a length of time until a specific number of new claims for a particular medication are processed; and/or a length of time until a number of new claims is processed that have a statically significant (such as a p-value of less than or equal to 0.25) difference with a previous WN. More generally, the time period may be long enough to capture a meaningful number of values, where both average, median and standard deviation can be calculated. This may allow the WN value to be updated if there are changes in either mean, median and standard deviation, e.g., when the center of ‘mass’ of the values has moved or a measure of dispersion of the values has changed.

Another approach may be patient-optimized and may be used to determine WN₁. Notably, for every medication N, a value UN may be selected such that historical data showing past adjudications is analyzed and a dynamic price may be calculated for each medication in quantity one, such that WN_{q} is less or equal to UN times the number of prescription transactions. For example, UN may be selected so that the derived WN_{q} is less than 80% of all other adjudicated prices for this medication. Table 1 presents a series of AN_{iq} values. Using these values and with UN equal to 80%, then WN_{q} may be $6.05.

TABLE 1

| | |
|---|---|
| 1 | 3.46 |
| 2 | 5.30 |
| 3 | 5.61 |
| 4 | 6.00 |
| 5 | 6.10 |
| 6 | 6.22 |
| 7 | 6.23 |
| 8 | 6.61 |
| 9 | 8.25 |
| 10 | 8.39 |
| 11 | 8.64 |
| 12 | 8.89 |
| 13 | 9.00 |
| 14 | 9.14 |
| 15 | 9.25 |
| 16 | 10.00 |
| 17 | 10.08 |
| 18 | 10.86 |
| 19 | 10.88 |
| 20 | 11.75 |

Yet another approach may be pharmacy-optimized. Notably, given CN_{iq} and a target margin M,

$${WN}_{q} = M + M \cdot {CN}_{iq} + {YN}_{q} + XN .$$

If M equals 5%, YN_{q} equals $1, and XN equals $1.50, then for a medication that cost the pharmacy $20, WN_{q}=$20+$20-5%+$1+$1.50=$23.50.

In some embodiments, the dynamic optimization may use one or more analytical techniques, including: Newton's method, an iterative technique, sequential quadratic programming, an interior point technique, a coordinate descent technique, a conjugate gradient technique, gradient descent, a subgradient technique, a bundle technique of descent, an ellipsoid technique, a conditional gradient technique, a quasi-Newton method, simultaneous perturbation stochastic approximation, a pattern search technique, an interpolation technique, a simplex technique, a genetic technique, a nonlinear optimization technique, and/or another optimization technique.

Moreover, in some embodiments, the dynamic optimization may be performed, at least in part, using a pretrained machine learning model or a pretrained neural network (such as a convolutional neural network, a generative adversarial network, a neural network with path memory, a long-short-term memory neural network, or another type of neural network). For example, the pretrained machine learning model or the pretrained neural network may be trained using a training dataset with historical information for previous transactions. Moreover, the pretrained machine learning model may be a classifier or a regression model, and may be trained using a supervised learning technique, such as: a support vector machine technique, a classification and regression tree technique, logistic regression, LASSO, linear regression, and/or another linear or nonlinear supervised-learning technique. Note that the pretrained machine learning model or the pretrained neural network may use the pharmacy information as inputs and may output information associated with the second potential prescription transaction and/or the comparison information.

While the preceding embodiments illustrated the prescription techniques as requesting selective approval (e.g., from a pharmacy technician), in other embodiments the prescription techniques occur in fully automated manner. Consequently, the selective approval may not be requested from a human. Instead. in these embodiments of the prescription techniques, a computer system (such as a computer system that is include in or associated with a pharmacy, e.g., a pharmacy management system) may automatically determine when there is a prescription-transaction advantage. Then, the computer system may automatically perform the prescription transaction. Otherwise, when there is not a prescription-transaction advantage, the computer system may not perform the prescription transaction. Thus, in these embodiments, the comparison information (operation 218 in FIG. 2) may not be performed.

In contrast, in existing pharmacy transactions, a pharmacy (or an associated computer system) may run a trial transaction based at least in part on insurance of a consumer or patient, and may tell or inform the consumer or patient of their copayment. Alternatively, a pharmacy (or an associated computer system) may run a trial transaction with a discount card of the consumer or patient. After a claim is processed, the computer system may store, in memory, the insurance or discount card information associated with the consumer or patient (e.g., in a profile of the consumer or patient). Note that when the consumer or patient returns to the pharmacy (such as during a subsequent visit to fill or renew a prescription), this stored information may be used to run a claim for the consumer or the patient. Alternatively, when the insurance or discount card information is manually entered, it can take, on average, three minutes for a pharmacy technician to perform a transaction to submit a claim for a prescription.

In the disclosed prescription techniques, for every claim, a computer system (such as a computer system associated with the pharmacy that executes software or program instructions that implement the prescription techniques) may reprice the claim and dynamically and automatically determine when there is a prescription advantage or cost savings. When there is a prescription advantage or cost savings, the computer system may automatically reverse another claim for this prescription (such as a claim that processes the prescription using insurance or a discount card associated with the consumer or patient, or payment based at least in part on the retail price or the Usual & Customary price). Then, the computer system may adjudicate a claim for the prescription using the prescription techniques. Otherwise, when there is not a prescription advantage, the computer system may not reverse the other claim and may not process a claim for the prescription using the prescription techniques.

Thus, in some embodiments, the prescription techniques may eliminate the need for human interaction when conducting a transaction for a prescription. Instead, the prescription techniques may allow the transaction to be conducted or adjudicated in less than 16 ms. Consequently, the prescription techniques may provide a significant reduction in the use of computer resources (such as processing time, memory accesses, network bandwidth, etc.) and pharmacy-staff time than the existing approaches described previously. For example, in addition to the manual processing time, in existing approaches it may take 1-2 s to perform a single comparison using a prescription benefits manager.

Moreover, the prescription techniques may provide additional revenue for pharmacies and cost savings for consumers or patients. For example, every discount card may have an associated administrative fee. When a prescription costs $20.00, there may be a $6.00 administrative fee when a pharmacy processes a claim using the pharmacy benefits manager associated with the discount card. Thus, the transaction for this prescription may net $14.00 to the pharmacy, while the pharmacy benefits manager associated with the discount card may claw back $6.00 for this transaction. Alternatively, when the prescription costs $30.00 and $20.00 is paid by the consumer (e.g., as their copayment), the pharmacy benefits manager may pay the pharmacy $10.00 (which may offset the administrative cost of $6.00). Consequently, the net for the pharmacy may be $24.00.

Alternatively, in the prescription techniques, when a claim to the aforementioned prescription is processed, $14.00 may go to the pharmacy. In addition, 20% of the 6.00 administrative fee may be paid to a provider of the prescription techniques. The remaining $4.80 may be split between the consumer or patient and the pharmacy. Thus, the consumer may pay $17.60 (instead of $20.00 in the existing approaches) and the pharmacy may receive an additional $2.40 net payment.

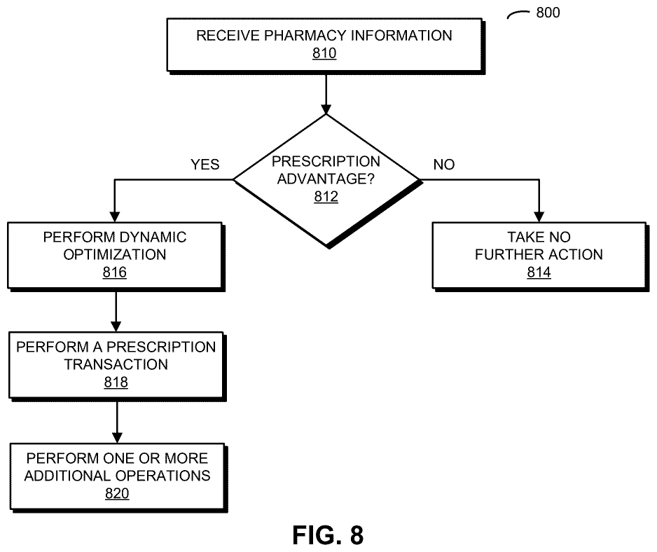

FIG. 8 presents a flow diagram illustrating an example of a method 800 for automatically and selectively performing a prescription transaction, which may be performed by a computer system, such as computer system 130 in FIG. 1. During operation, the computer system may receive pharmacy information (operation 810), where the pharmacy information specifies, for a potential prescription transaction for a prescription, a cost for an individual, an overhead associated with an entity when the potential prescription transaction is facilitated by the entity, and a residual amount.

Note that the cost and the overhead may be predefined and may be associated with a PBM that manages prescription benefits. Moreover, the pharmacy information may specify a potential prescription cost corresponding to a drug ingredient in the prescription and potential revenue for a pharmacy for the potential prescription transaction.

Then, the computer system may assess whether there is a prescription-transaction advantage (operation 812) based at least in part on the overhead and a second overhead associated with a second entity when a second potential prescription transaction for the prescription is facilitated by the second entity. When there is no prescription-transaction advantage (operation 812), the computer system may take no further action (operation 814). Alternatively, when there is the prescription-transaction advantage (operation 812), the computer system may perform dynamically optimization (operation 816). For example, the dynamic optimization, based at least in part on the prescription-transaction advantage and one or more prescription-transaction constraints, may determine a second cost for the individual and a second residual amount associated with the second potential prescription transaction.

Note that the entity may be different from the pharmacy and may be different from the second entity. For example, the entity may be a PBM or a provider of a prescription discount card for the prescription and the second entity may be a second PBM or a second provider of a second prescription discount card for the prescription.

Moreover, the second computer system may be associated with the pharmacy. For example, the pharmacy may have a contract with the second entity for a service that determines when there is the prescription-transaction advantage.

Furthermore, the prescription-transaction advantage may correspond to or may be a function of a difference of the overhead and the second overhead, and there may be the prescription-transaction advantage when the overhead is greater than the second overhead (and, thus, when the second entity is able to or willing to accept a smaller amount in the second potential prescription transaction than the entity is able to or willing to accept in the potential prescription transaction).

Additionally, the one or more prescription-transaction constraints may be associated with the pharmacy. For example, the pharmacy (or a corporation that owns multiple pharmacies, which include the pharmacy) may provide (e.g., to the computer system) or specify the one or more prescription-transaction constraints. In some embodiments, the one or more prescription-transaction constraints may be dynamically varied by the pharmacy, such as from one individual to another or for the individual as a function of time (such as based at least in part on the historical information described further below).

The dynamic optimization may segment the prescription-transaction advantage into a portion and a second portion, and the second cost may correspond to (or be a function of) a sum of the cost and the portion. In some embodiments, the one or more prescription-transaction constraints may include a minimum potential revenue of a pharmacy. Alternatively or additionally, the one or more prescription-transaction constraints may include: a preference for maximizing a potential revenue, a second preference for minimizing the second cost, or a third preference for apportioning a portion of the prescription-transaction advantage to the second cost and apportioning a second portion of the prescription-transaction advantage to the potential revenue. Thus, depending on the preferences of the pharmacy (as specified by the one or more prescription-transaction constraints), the computer system may dynamically optimize or tailor the second potential prescription transaction to the advantage of the pharmacy, the advantage of the individual or any dynamic point in between.

Next, when there is the prescription-transaction advantage (operation 812), the computer system automatically and selectively performs the prescription transaction (operation 818) corresponding to the second potential prescription transaction with the second entity and using the second overhead. This may include providing the prescription to the individual.

Furthermore, the prescription transaction is associated with second potential revenue for the pharmacy. In some embodiments, the one or more prescription-transaction constraints may include that the second potential revenue is positive.

In some embodiments, the computer system may optionally perform one or more additional operations (operation 820). For example, the computer system may access historical information specifying prior prescription transactions for prescriptions of at least the individual, and the dynamic optimizing may be based at least in part on the historical information.

Moreover, the computer system may receive at least one of the one or more prescription-transaction constraints associated with the second computer system. For example, the pharmacy information may specify at least one of the one or more prescription-transaction constraints.

Furthermore, the computer system may: receive acceptance information associated with the second computer system, where the acceptance information specifies that a prescription transaction for the prescription has been conducted between the pharmacy and the individual; and selectively perform a second prescription transaction based at least in part on the acceptance information, where the second prescription transaction includes receiving the second overhead.

In some embodiments of method 200 (FIG. 2), 300 (FIG. 3) and/or 800, there may be additional or fewer operations. Furthermore, the order of the operations may be changed, and/or two or more operations may be combined into a single operation.

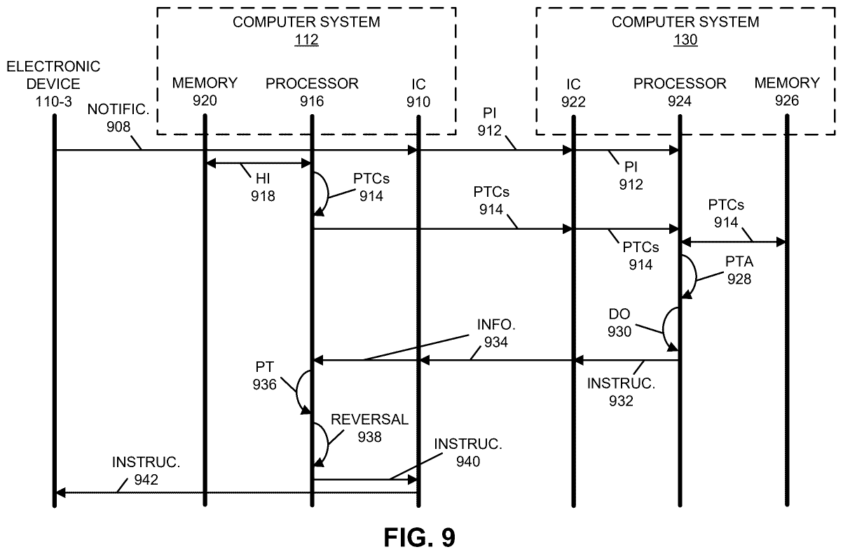

Embodiments of the prescription techniques are further illustrated in FIG. 9, which presents a drawing illustrating an example of communication among electronic device 110-3, computer system 112 and computer system 130. In FIG. 9, an interface circuit (IC) 910 in computer system 112 may provide pharmacy information (PI) 912 to computer system 130, where the pharmacy information specifies, for a potential prescription transaction for a prescription, a cost for an individual, an overhead associated with an entity when the potential prescription transaction is facilitated by the entity, and a residual amount. For example, computer system 112 may provide pharmacy information 912 when the individual is at a point-of-sale terminal in a pharmacy associated with computer system 112, such as when the individual is filling or picking up the prescription at the pharmacy (and, thus, when the individual is conducting or is about to conduct a prescription transaction). Notably, computer system 112 may provide pharmacy information 912 based at least in part on a notification 908 received from electronic device 110-3, such as the point-of-sale terminal in the pharmacy. This notification may include information that specifies a prescription drug benefit associated with the individual (such as a health-insurance policy, an associated PBM, etc.) and a prescription associated with the individual. Alternatively, in some embodiments, computer system 112 may provide pharmacy information 912 in advance, such as after a prescription is submitted to the pharmacy but before the individual picks up the prescription at or from the pharmacy.

In some embodiments, interface circuit 910 optionally provides one or more prescription-transaction constraints (PTCs) 914 to computer system 130. The one or more prescription-transaction constraints 914 may be associated with the pharmacy. Note that the one or more prescription-transaction constraints 914 may be provided in advance of the individual filling the prescription at the pharmacy. Alternatively, the one or more prescription-transaction constraints 914 may be provided while the individual is filling the prescription or conducting the prescription transaction. In these embodiments, the one or more prescription-transaction constraints 914 may be dynamically determined by a processor 916 in computer system 112, such as based at least in part on historical information (HI) 918 about previous prescription transactions of at least the individual, which may be stored in memory 920 in or associated with computer system 112.

After receiving pharmacy information 912 and/or the one or more prescription-transaction constraints 914, interface circuit 922 in computer system 130 may provide this information to processor 924 in computer system 130. Alternatively or additionally, processor 924 may access at least one of the one or more prescription-transaction constraints 914 in memory 926 in computer system 130.

Then, processor 924 may assess whether there is a prescription-transaction advantage (PTA) 928 based at least in part on the overhead and a second overhead associated with a second entity when a second potential prescription transaction for the prescription is facilitated by the second entity. When there is the prescription-transaction advantage 928, processor 924 may dynamically optimize (DO) 930, based at least in part on the prescription-transaction advantage and one or more prescription-transaction constraints, a second cost for the individual and a second residual amount associated with the second potential prescription transaction. In general, dynamic optimization 930 may be over one or more variables. For example, dynamic optimization 930 may attempt to maximize a revenue of the pharmacy, while minimizing a cost to the individual.

Next, when there is the prescription-transaction advantage 928, processor 924 may instruct 932 interface circuit 922 to provide information 934 associated with the second potential prescription transaction to computer system 112, where information 934 specifies the second cost and the second residual amount.

After receiving information 934, interface circuit 910 may provide information 934 to processor 916. Then, based at least in part on information 934, processor 916 may automatically and selectively perform prescription transaction (PT) 936. Prescription transaction 936 for the prescription between the pharmacy and the individual may correspond to the second potential prescription transaction with the second entity and may use the second overhead. Note that performing 936 the prescription transaction may include: providing a reversal 938 of the potential prescription transaction; and providing an instruction 940 to interface circuit 910 to provide an instruction 942 to electronic device 110-3 for the staff at the pharmacy to collect the payment from the individual (including the second cost).

While FIGS. 4 and 9 illustrate communication between components using unidirectional or bidirectional communication with lines having single arrows or double arrows, in general the communication in a given operation in this figure may involve unidirectional or bidirectional communication. Moreover, while FIGS. 4 and 9 illustrate operations being performed sequentially or at different times, in other embodiments at least some of these operations may, at least in part, be performed concurrently or in parallel.

We now describe embodiments of an electronic device, which may perform at least some of the operations in the prescription techniques. FIG. 10 presents a block diagram illustrating an example of an electronic device 1000 in accordance with some embodiments, such as one of: base station 108, one of electronic devices 110, computer system 112, one of access points 116, radio node 118, switch 128, and/or computer system 130. This electronic device includes processing subsystem 1010, memory subsystem 1012, and networking subsystem 1014. Processing subsystem 1010 includes one or more devices configured to perform computational operations. For example, processing subsystem 1010 can include one or more microprocessors, graphics processing units (GPUs), ASICs, microcontrollers, programmable-logic devices, and/or one or more digital signal processors (DSPs).

Memory subsystem 1012 includes one or more devices for storing data and/or instructions for processing subsystem 1010 and networking subsystem 1014. For example, memory subsystem 1012 can include DRAM, static random access memory (SRAM), and/or other types of memory. In some embodiments, instructions for processing subsystem 1010 in memory subsystem 1012 include: one or more program modules or sets of instructions (such as program instructions 1022 or operating system 1024, such as Linux, UNIX, Windows Server, or another customized and proprietary operating system), which may be executed by processing subsystem 1010. Note that the one or more computer programs, program modules or instructions may constitute a computer-program mechanism. Moreover, instructions in the various modules in memory subsystem 1012 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 1010.

In addition, memory subsystem 1012 can include mechanisms for controlling access to the memory. In some embodiments, memory subsystem 1012 includes a memory hierarchy that comprises one or more caches coupled to a memory in electronic device 1000. In some of these embodiments, one or more of the caches is located in processing subsystem 1010.

In some embodiments, memory subsystem 1012 is coupled to one or more high-capacity mass-storage devices (not shown). For example, memory subsystem 1012 can be coupled to a magnetic or optical drive, a solid-state drive, or another type of mass-storage device. In these embodiments, memory subsystem 1012 can be used by electronic device 1000 as fast-access storage for often-used data, while the mass-storage device is used to store less frequently used data.

Networking subsystem 1014 includes one or more devices configured to couple to and communicate on a wired and/or wireless network (i.e., to perform network operations), including: control logic 1016, an interface circuit 1018 and one or more antennas 1020 (or antenna elements). (While FIG. 10 includes one or more antennas 1020, in some embodiments electronic device 1000 includes one or more nodes, such as antenna nodes 1008, e.g., a metal pad or a connector, which can be coupled to the one or more antennas 1020, or nodes 1006, which can be coupled to a wired or optical connection or link. Thus, electronic device 1000 may or may not include the one or more antennas 1020. Note that the one or more nodes 1006 and/or antenna nodes 1008 may constitute input(s) to and/or output(s) from electronic device 1000.) For example, networking subsystem 1014 can include a Bluetooth™ networking system, a cellular networking system (e.g., a 3G/4G/5G network such as UMTS, LTE, etc.), a universal serial bus (USB) networking system, a coaxial interface, a High-Definition Multimedia Interface (HDMI) interface, a networking system based on the standards described in IEEE 802.11 (e.g., a Wi-Fi® networking system), an Ethernet networking system, and/or another networking system.

Networking subsystem 1014 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, electronic device 1000 may use the mechanisms in networking subsystem 1014 for performing simple wireless communication between the electronic devices, e.g., transmitting advertising or beacon frames and/or scanning for advertising frames transmitted by other electronic devices as described previously.

Within electronic device 1000, processing subsystem 1010, memory subsystem 1012, and networking subsystem 1014 are coupled together using bus 1028. Bus 1028 may include an electrical, optical, and/or electro-optical connection that the subsystems can use to communicate commands and data among one another. Although only one bus 1028 is shown for clarity, different embodiments can include a different number or configuration of electrical, optical, and/or electro-optical connections among the subsystems.

In some embodiments, electronic device 1000 includes a display subsystem 1026 for displaying information on a display, which may include a display driver and the display, such as a liquid-crystal display, a multi-touch touchscreen, etc.

Moreover, electronic device 1000 may include a user-interface subsystem 1030, such as: a mouse, a keyboard, a trackpad, a stylus, a voice-recognition interface, and/or another human-machine interface. In some embodiments, user-interface subsystem 1030 may include or may interact with a touch-sensitive display in display subsystem 1026.

Electronic device 1000 can be (or can be included in) any electronic device with at least one network interface. For example, electronic device 1000 can be (or can be included in): a desktop computer, a laptop computer, a subnotebook/netbook, a server, a tablet computer, a cloud-based computing system, a point-of-sale terminal (such as a cash register), a smartphone, a cellular telephone, a smartwatch, a wearable electronic device, a consumer-electronic device, a portable computing device, an access point, a transceiver, a router, a switch, communication equipment, an eNodeB, a controller, test equipment, and/or another electronic device.

Although specific components are used to describe electronic device 1000, in alternative embodiments, different components and/or subsystems may be present in electronic device 1000. For example, electronic device 1000 may include one or more additional processing subsystems, memory subsystems, networking subsystems, and/or display subsystems. Additionally, one or more of the subsystems may not be present in electronic device 1000. Moreover, in some embodiments, electronic device 1000 may include one or more additional subsystems that are not shown in FIG. 10. Also, although separate subsystems are shown in FIG. 10, in some embodiments some or all of a given subsystem or component can be integrated into one or more of the other subsystems or component(s) in electronic device 1000. For example, in some embodiments instructions 1022 is included in operating system 1024 and/or control logic 1016 is included in interface circuit 1018.

Moreover, the circuits and components in electronic device 1000 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit (which is sometimes referred to as a ‘communication circuit’) may implement some or all of the functionality of networking subsystem 1014 and/or of electronic device 1000. The integrated circuit may include hardware and/or software mechanisms that are used for transmitting wireless signals from electronic device 1000 and receiving signals at electronic device 1000 from other electronic devices. Aside from the mechanisms herein described, radios are generally known in the art and hence are not described in detail. In general, networking subsystem 1014 and/or the integrated circuit can include any number of radios. Note that the radios in multiple-radio embodiments function in a similar way to the described single-radio embodiments.

In some embodiments, networking subsystem 1014 and/or the integrated circuit include a configuration mechanism (such as one or more hardware and/or software mechanisms) that configures the radio(s) to transmit and/or receive on a given communication channel (e.g., a given carrier frequency). For example, in some embodiments, the configuration mechanism can be used to switch the radio from monitoring and/or transmitting on a given communication channel to monitoring and/or transmitting on a different communication channel. (Note that ‘monitoring’ as used herein comprises receiving signals from other electronic devices and possibly performing one or more processing operations on the received signals)

In some embodiments, an output of a process for designing the integrated circuit, or a portion of the integrated circuit, which includes one or more of the circuits described herein may be a computer-readable medium such as, for example, a magnetic tape or an optical or magnetic disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII) or Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While the preceding discussion used Wi-Fi, LTE and/or Ethernet communication protocols as illustrative examples, in other embodiments a wide variety of communication protocols and, more generally, communication techniques may be used. Thus, the prescription techniques may be used in conjunction with a variety of network interfaces. Furthermore, while some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the prescription techniques may be implemented using program instructions 1022, operating system 1024 (such as a driver for interface circuit 1018) or in firmware in interface circuit 1018. Alternatively or additionally, at least some of the operations in the prescription techniques may be implemented in a physical layer, such as hardware in interface circuit 1018.

Note that the use of the phrases ‘capable of,’ ‘capable to,’ ‘operable to,’ or ‘configured to’ in one or more embodiments, refers to some apparatus, logic, hardware, and/or element designed in such a way to enable use of the apparatus, logic, hardware, and/or element in a specified manner.

While examples of numerical values are provided in the preceding discussion, in other embodiments different numerical values are used. Consequently, the numerical values provided are not intended to be limiting.

In the preceding description, we refer to ‘some embodiments.’ Note that ‘some embodiments’ describes a subset of all of the possible embodiments, but does not always specify the same subset of embodiments.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A computer system, comprising:
an interface circuit;
a processor coupled to the interface circuit;
memory, coupled to the processor, configured to store program instructions, wherein, when executed by the processor, the program instructions cause the computer system to automatically and selectively perform a prescription transaction by performing operations comprising:
receiving or accessing pharmacy information, wherein the pharmacy information specifies, for a potential prescription transaction for a prescription, a cost for an individual, an overhead associated with an entity when the potential prescription transaction is facilitated by the entity, and a residual amount;
assessing whether there is a prescription-transaction advantage based at least in part on the overhead and a second overhead associated with a second entity when a second potential prescription transaction for the prescription is facilitated by the second entity;
when there is the prescription-transaction advantage, dynamically optimizing, based at least in part on the prescription-transaction advantage and one or more prescription-transaction constraints, a second cost for the individual and a second residual amount associated with the second potential prescription transaction; and
when there is the prescription-transaction advantage, automatically and selectively performing the prescription transaction corresponding to the second potential prescription transaction with the second entity and using the second overhead.

2. The computer system of claim 1, wherein the cost and the overhead are predefined and are associated with a prescription benefits manager (PBM) that manages prescription benefits.

3. The computer system of claim 1, wherein the pharmacy information specifies a potential prescription cost corresponding to a drug ingredient in the prescription and potential revenue for a pharmacy for the potential prescription transaction; and
wherein the prescription transaction is associated with second potential revenue for the pharmacy.

4. The computer system of claim 3, wherein the one or more prescription-transaction constraints comprise that the second potential revenue is positive.

5. The computer system of claim 1, wherein the entity is different from a pharmacy and is different from the second entity.

6. The computer system of claim 1, wherein the entity is a prescription benefits manager (PBM) or a provider of a prescription discount card for the prescription and the second entity is a second PBM or a second provider of a second prescription discount card for the prescription.

7. The computer system of claim 1, wherein the computer system is associated with a pharmacy.

8. The computer system of claim 7, wherein the pharmacy has a contract with the second entity for a service that determines when there is the prescription-transaction advantage.

9. The computer system of claim 1, wherein the operations comprise accessing historical information specifying prior prescription transactions for prescriptions of at least the individual; and
wherein the dynamic optimizing is based at least in part on the historical information.

10. The computer system of claim 1, wherein the operations comprise receiving or accessing at least one of the one or more prescription-transaction constraints.

11. The computer system of claim 1, wherein the pharmacy information specifies at least one of the one or more prescription-transaction constraints.

12. The computer system of claim 1, wherein the operations comprise:
receiving acceptance information, wherein the acceptance information specifies that the prescription transaction for the prescription will be conducted between a pharmacy and the individual; and
wherein the selective performing of the prescription transaction is based at least in part on the acceptance information.

13. The computer system of claim 1, wherein the prescription-transaction advantage corresponds to a difference of the overhead and the second overhead; and
wherein there is the prescription-transaction advantage when the overhead is greater than the second overhead.

14. The computer system of claim 1, wherein the performing of the prescription transaction comprises providing the prescription to the individual.

15. The computer system of claim 1, wherein the one or more prescription-transaction constraints are associated with a pharmacy.

16. The computer system of claim 1, wherein the dynamic optimization segments the prescription-transaction advantage into a portion and a second portion, and the second cost corresponds to a sum of the cost and the portion.

17. The computer system of claim 1, wherein the one or more prescription-transaction constraints comprise a minimum potential revenue of a pharmacy.

18. The computer system of claim 1, wherein the one or more prescription-transaction constraints comprise: a preference for maximizing a potential revenue, a second preference for minimizing the second cost, or a third preference for apportioning a portion of the prescription-transaction advantage to the second cost and apportioning a second portion of the prescription-transaction advantage to the potential revenue.

19. A non-transitory computer-readable storage medium for use in conjunction with a computer system, the computer-readable storage medium configured to store program instructions that, when executed by the computer system, causes the computer system to automatically and selectively performing a prescription transaction by performing operations comprising:
receiving pharmacy information, wherein the pharmacy information specifies, for a potential prescription transaction for a prescription, a cost for an individual, an overhead associated with an entity when the potential prescription transaction is facilitated by the entity, and a residual amount;
assessing whether there is a prescription-transaction advantage based at least in part on the overhead and a second overhead associated with a second entity when a second potential prescription transaction for the prescription is facilitated by the second entity;
when there is the prescription-transaction advantage, dynamically optimizing, based at least in part on the prescription-transaction advantage and one or more prescription-transaction constraints, a second cost for the individual and a second residual amount associated with the second potential prescription transaction; and
when there is the prescription-transaction advantage, automatically and selectively performing the prescription transaction corresponding to the second potential prescription transaction with the second entity and using the second overhead.

20. A method for automatically and selectively performing a prescription transaction, comprising:
by a computer system:
receiving pharmacy information, wherein the pharmacy information specifies, for a potential prescription transaction for a prescription, a cost for an individual, an overhead associated with an entity when the potential prescription transaction is facilitated by the entity, and a residual amount;
assessing whether there is a prescription-transaction advantage based at least in part on the overhead and a second overhead associated with a second entity when a second potential prescription transaction for the prescription is facilitated by the second entity;
when there is the prescription-transaction advantage, dynamically optimizing, based at least in part on the prescription-transaction advantage and one or more prescription-transaction constraints, a second cost for the individual and a second residual amount associated with the second potential prescription transaction; and
when there is the prescription-transaction advantage, automatically and selectively performing the prescription transaction corresponding to the second potential prescription transaction with the second entity and using the second overhead.
